(19) United States
(12) Patent Application Publication
(10) Pub. No.: US 2002/0114830 A1
(43) Pub. Date: Aug. 22, 2002

(54) Novel compostion and process

(76) Inventor: Maarten Van Den Braak, Coleford (GB)

Correspondence Address: GLAXOSMITHKLINE, Corporate Intellectual Property - UW2220, P.O. Box 1539, King of Prussia, PA, 19406-0939, US

(21) Appl. No.: 09/975,767

(22) Filed: Oct. 11, 2001

Related U.S. Application Data
(63) Continuation-in-part of application No. 09/381,259, filed on Dec. 10, 1999, now abandoned.
Application No. 09/381,259 is a 371 of international application No. PCT/EP98/10580, filed on Mar. 13, 1998.
Application No. 09/381,259 is a 371 of international application No. 09/825,433, filed on Apr. 2, 2001.
Application No. 09/825,433 is a division of application No. 09/043,373, filed on Aug. 7, 1998.
Application No. 09/043,373 is a 371 of international application No. PCT/EP96/04168, filed on Sep. 19, 1996.

(30) Foreign Application Priority Data
Mar. 20, 1997 (GB) 9705813.5

(51) Int. Cl. A61K 9/64; A61K 35/78

(52) U.S. Cl. 424/456; 424/776

(57) ABSTRACT

Essentially non-aqueous compositions suitable for oral administration comprising a biologically active oil, or a biologically active oil or an oil-soluble ingredient as a dispersion in a suitable carrier oil, dispersed in a physiologically acceptable emulsifier or emulsifier mixture having an HLB (hydrophilic/lipophilic balance) value of between 10 and 18 and encapsulated in a capsule material that is soluble in gastric juice, their production and use in the maintenance and/or promotion of health.

### RELATED INFORMATION

This is a continuation-in-part application of U.S. Ser. No. 09/381,259, filed Dec. 10, 1999 which is the §371 national stage entry of PCT/EP98/10580, filed Mar. 13, 1998 and U.S. Ser. No. 09/825,433, filed Apr. 2, 2001 which is a divisional of U.S. Ser. No. 09/043,373, (now U.S. Pat. No. 6,251,441) filed Aug. 7, 1998 which is the §371 national stage entry of PCT/EP96/04168, filed Sep. 19, 1996.

### FIELD OF THE INVENTION

The invention relates to compositions suitable for oral administration comprising oils or oil soluble ingredients useful in the maintenance and/or promotion of health, in capsule form.

The present invention provides an essentially non-aqueous composition for use in human or animal healthcare comprising a biologically active oil or an oil-soluble ingredient or a biologically active oil or an oil-soluble ingredient as a dispersion in a suitable carrier oil, dispersed in a physiologically acceptable emulsifier or emulsifier mixture having an HLB (hydrophilic/lipophilic balance) value of between 10 and 18 and encapsulated in a capsule material that is soluble in gastric juice.

WO94/06310 discloses an aqueous composition for the preparation of optically clear products for use in human and animal healthcare comprising 0.1 to 2.0% w/w of an oil soluble ingredient as a 20-30% w/w dispersion in a suitable oil or 0.1 to 5.0% w/v as the pure crystalline ingredient, 2-20% w/w of an emulsifier having an HLB value of between 10 and 18 or where a blend of emulsifiers is employed, a calculated HLB value of between 10 and 18 and 0.1% w/w of an antioxidant or a mixture of antioxidants.

WO 95/24832 discloses similar aqueous compositions for the preparation of optically clear products based on biologically active oils.

Techniques for preparing biologically active oil or oil-soluble ingredient compositions are described in WO 94/06310 and WO 95/24832 and non-aqueous encapsulated compositions may be prepared using the same techniques but omitting the addition of water to the compositions described in those documents. The entire disclosures of WO 94/06310 and WO 95/24832 are incorporated herein by reference.

Thus, referring to WO 94/06310, an essentially non-aqueous composition for encapsulation may be prepared by:

a) dispersing an antioxidant in an emulsifier or mixture of emulsifiers having an HLB (hydrophilic/lipophilic balance) value of between 10-18 while heating to a temperature of approximately 40° C.;

b) dispersing one or more oil-soluble ingredients, or one or more oil-soluble ingredients as a dispersion in a suitable carrier oil, in the mixture in a) above while heating to between about 80-200° C. so as to yield a transparent mixture.

Alternatively, referring to WO 95/24832, an essentially non-aqueous composition for encapsulation may be prepared by:

a) mixing a biologically active oil with an antioxidant or antioxidant mixture

b) dispersing the oil-antioxidant mixture in an emulsifier or mixture of emulsifiers having an HLB value of from 10 to 18, while heating to between 50-150° C. so as to yield a transparent mixture. Optionally the antioxidant in a) is dispersed initially with the emulsifier before mixing with the biologically active oil.

In “Microemulsions Theory and Practice” (Edited Leon M Prince chapter 3, Academic Press Inc, N.Y., 1977), it is stated that stable microemulsions result when an emulsifier is chosen that is chemically matched to the oil. Also in order to form satisfactory emulsions it can be helpful to use two kinds of emulsifier, a primary surfactant and a cosurfactant.

WO 97/10725 provides compositions obtained by using an emulsifier mixture comprising fatty acid residues which chemically matches the fatty acid residues of the oil or of the oil or oil-soluble ingredient as an oil dispersion using specific ratios of oil or oil dispersion to emulsifier and specific ratios of primary to cosurfactant.

WO 97/10725 provides a process for preparing an aqueous composition having enhanced bioavailability for human or animal healthcare comprising a) mixing 0.001-2.0% of an oil soluble ingredient or 0.1-2.0% w/w of an oil soluble ingredient as a 20-30% dispersion in a suitable oil with 2-10% of an emulsifier mixture having an HLB (hydrophilic lipophilic balance) value of between 10 and 18 and heating to between 25 and 150° C. so as to yield a transparent mixture; and b) combining the mixture with water while continuously stirring to provide a transparent composition. The emulsifier mixture is a combination of a primary surfactant and a secondary or cosurfactant, wherein the fatty acid profile of the emulsifier mixture matches the fatty acid profile of the oil components of the composition, the HLB of the primary surfactant is greater than the cosurfactant and the weight ratio of oil or oil dispersion to emulsifier is between 1:1 and 1:7 and the ratio of primary to cosurfactant is between 10:1 and 200:1.

An essentially non-aqueous composition for encapsulation is prepared using the techniques described in WO 97/10725 by mixing a biologically active oil or a biologically active oil or oil soluble ingredient as a dispersion in a suitable carrier oil, with an emulsifier mixture having an HLB (hydrophilic lipophilic balance) value of between 10 and 18, heating to between 25 and 150° C., if required, so as to yield a transparent mixture and, where heat has been applied, cooling the said transparent mixture to room temperature.

Using the techniques of WO 97/10725 the entire content of which is incorporated herein by reference, the emulsifier mixture is a combination of a primary surfactant and a secondary surfactant or cosurfactant, wherein the fatty acid profile of the emulsifier mixture matches the fatty acid profile of the oil or oil dispersion of the oil or oil soluble ingredient and the HLB of the primary surfactant is greater than that of the cosurfactant.

Typically a composition for encapsulation according to the invention is prepared by mixing 0.15-50% w/w of a biologically active oil, or a biologically active oil or oil soluble ingredient as a dispersion in a suitable carrier oil, with 50-99.85% w/w of emulsifier until a transparent mixture is formed, if necessary at elevated temperature and with subsequent cooling. The weight ratio of total oil or oil soluble ingredient to emulsifier is preferably between 1:1 and 1:7 and, where a cosurfactant is present, the ratio of primary surfactant to cosurfactant is preferably between 10:1 and 200:1. In some cases it is also desirable to include a polymeric alcohol such as polyethylene glycol. This has been found to be particularly beneficial for preparing formulations containing tocopheryl acetate. In this case the weight ratio of primary surfactant to polymeric alcohol is between 1:1 and 1:10.

Preferably the ratio of total oil to emulsifier is between 1:1 and 1:5. Preferably the ratio of primary surfactant to cosurfactant (when present) is between 20:1 and 50:1, and is most suitably about 30:1.

Surprisingly, it has been found that including a small amount of a cosurfactant allows less overall emulsifier to be used. This is particularly valuable where reducing emulsifier content has cost and taste benefits and reduces the amounts of ‘synthetic’ components in compositions for oral administration that are based on natural ingredients.

The material used for encapsulation is typically a water-soluble material such as gelatin. Soft gelatin capsules are preferred. When swallowed by a human or animal, the capsule disintegrates in the stomach, releasing the oil or oil-soluble ingredient/emulsifier composition for emulsification in the gastric juice and making the oil or oil-soluble ingredient available for absorption. Using the system of WO 97/10725 assists in the formation of emulsions with increased bioavailability.

Encapsulation of the oil or oil-soluble ingredient/emulsifier composition is carried out conventionally, for example using procedures described in The Pharmaceutical CODEX, Twelfth Edition, Ed Walter Lund, P23-24, 1994.

The encapsulated composition of the present invention is a product with desirable properties, particularly high dispersability in the stomach following oral administration, using ingredients which have hitherto been found to be difficult to solubilise satisfactorily in this kind of product. Further advantages of an encapsulated formulation over the aqueous compositions of the prior art include the convenience of a low volume, solid dosage form and the avoidance of any undesirable taste which may be associated with any of the ingredients when administered in a liquid dosage form.

An important advantage of the compositions of the present invention over known compositions is one of economy since it allows products with minimal amounts of emulsifiers to be developed. The compositions are particularly useful for incorporation of biologically active materials into preparations that result in a microdispersed form in the stomach which facilitates uptake. Certain biologically active oils and oil soluble ingredients are vitamins and provitamins such as vitamins A, D, E, carotenoid pigments and nutritionally important fatty acids. By biologically active oil is meant natural or synthetic oils which are or contain biologically active ingredients in particular prophylactic or therapeutic agents.

Preferably biologically active oils are oils of natural origin for example from the seeds or flowers of the Ribes, Boraginaceae, Labiataea, Onagraceae and Curcubitaceae species, oils of fungal origin, fish oils or other natural oils. Preferred oils include evening primrose oil, borage/starflower oil and blackcurrant seed oil.

Suitably compositions of the invention contain a total amount of oil in the range 0.15-50% , preferably 10-50% by weight. Oils for use in the present invention can be extracted from natural sources by processes known in the art. Oils are commercially available, for example, from Sigma Chemical Co., Poole, Dorset. The compositions can contain more than one biologically active oil.

Other biologically active ingredients suitable for use in the capsule composition according to the invention include carotenoid pigments, eg. β-carotene and oil soluble vitamins, eg. tocopherols such as tocopherol acetate (vitamin E).

Compositions containing carotenoids, such as lycopene and β-carotene as an oil soluble ingredient are particularly useful. Particularly suitable sources of β-carotene include both natural and synthetic β-carotene as dispersions in oil (as available from various commercial sources including those mentioned herein).

The biologically active oils may be and the oil-soluble ingredients are mixed or dispersed with other suitable oils in particular, consumable oils for example, corn, peanut, safflower, olive and rapeseed oils as well as many essential oils.

Emulsifiers for use in compositions of the invention may be any anionic, cationic, amphoteric or non-ionic emulsifiers which are suitable for consumption by humans or animals. Preferably the emulsifiers are non-ionic emulsifiers or mixtures of emulsifiers having an HLB (hydrophilic/lipophilic balance) of 12-16 and most preferably have an HLB value of 15. Suitable emulsifiers include Tween 60 (polyoxyethylene(20)sorbitan monostearate, Tween 40 (polyoxyethylene(20)-sorbitan monopalmitate), Tween 80 (polyoxyethylenesorbitan monooleate) and Span 80 (sorbitan monooleate) available from ICI Speciality Chemicals, Leatherhead, Surrey or from Sigma Chemical Company, Poole, Dorset. Preferred emulsifier mixtures include mixtures of Tween 80 and Span 80 or Tween 80 and Tween 40.

Suitably the emulsifier mixture is a binary or tertiary blend of emulsifiers, for example blends of Tween 60 with a sucrose ester emulsifier (manufactured by Mitsubishi Kasei Food Corporation, Ichikawa Building, 13-3 Ginza 5-Chome, Chuo-ku, Tokyo 104, Japan) or blends of Tween 60 and sucrose ester and a polyglycerol ester of a fatty acid (available from Grindsted Products Limited., Northern Way, Bury St. Edmunds, Suffolk).

The amount of emulsifier or emulsifier mixture in the composition is selected as an amount which will vary depending upon which specific biologically active oil, or oil or oil-soluble ingredient dispersion is used, its method of preparation, and how much is included.

The composition advantageously comprises in addition an antioxidant which can be for example, α-tocopherol, ascorbic acid, ascorbyl palmitate, butylated hydroxyanisole (BHA) and butylated hydroxytoluene (BHT) or a mixture of such antioxidants. Particularly preferred antioxidants are α-tocopherol, ascorbyl palmitate and ascorbic acid.

Compositions of the present invention containing biologically active oils and oil-soluble ingredients are believed to disperse in a micellar form or as microemulsions in an aqueous environment because they exhibit certain characteristics eg. transparency when viewed by transmitted light in test dissolutions. Therefore, a further advantage of the capsule compositions according to the present invention is that the fine dispersion of these oils and oil-soluble ingredients in the stomach will help to promote their efficient uptake by body tissues when the composition is administered orally as a capsule. While the small particle size of the particles of biologically active oils and oil soluble ingredients favour their uptake, the simultaneous presentation or ingestion of these materials with an emulsifier will also encourage efficient transfer of these substances across membranes.

The dispersed microemulsions also have acid resistance. This is advantageous because prior to absorption from the intestinal tract, the microemulsion is able to survive the strongly acid conditions of the stomach.

In a further aspect of the invention there is provided a method of administration of a biologically active oil or an oil-soluble material to a human or animal by oral administration of a capsule composition according to the invention. The invention also provides a method of improving the bioavailability of a biologically active oil or oil-soluble ingredient in a human or animal body comprising orally administering a capsule composition according to the invention.

Optionally the compositions also contain antioxidant cofactors such as zinc, selenium and manganese which are needed for the body's naturally occurring antioxidant enzymes. Further nutritive ingredients may be added, such as other vitamins and minerals as described in “The Food Labelling Regulations 1984” Statutory Instrument No. 1305 (1984) H.M.S.O., London.

Suitably processing aids can be incorporated. Such aids may include ingredients which influence pH, redox potential, enzyme activity, hydrogen bonding and/or other aspects. Processing aids are for example sulphur dioxide, other antioxidants, metal salts, acids (eg. phosphoric and citric acid), alkalis, surfactants such as lecithin and starch plasticisers eg. calcium chloride.

Ingredients which may be subject to a loss of nutritional value are suitably added at a late stage of the process. Optionally the product can be produced in light or oxygen excluding containers prior to encapsulation to increase protection of materials sensitive to light or oxygen induced degradation.

The use of high temperatures when preparing compositions for encapsulation incurs the risk of degradation of certain oils or oil-soluble ingredients unless suitable precautions are taken. For example it may be desirable to incorporate an antioxidant in the initial stages of preparation or to exclude oxygen by heating the mixture in an atmosphere of nitrogen.

In a preferred aspect of the invention there is provided the use of carotene and tocopherol (including active derivatives thereof) capsules to deliver high bioavailability carotenes and Vitamin E by oral administration for use in medicine.

The invention is illustrated by the following Examples. The improved bioavailability of materials delivered by capsule compositions of this invention can be assessed by the test regime set out after the Examples.

### EXAMPLE 1

| | % w/w |
|---|---|
| β-carotene (30% dispersion) | 12.5 |
| emulsifier (polysorbate 80) | 25 |
| emulsifier (polysorbate 40) | 50 |
| tocopheryl acetate | 12.5 |

The β-carotene and tocopheryl acetate are dispersed in the two emulsifiers and the mixture is heated to 140° C. with stirring. At this point the mixture should remain transparent. Finally, the mixture is cooled rapidly to room temperature. The resultant mixture is encapsulated in gelatin capsules so as to contain approx. 15 mg β-carotene per capsule.

### EXAMPLE 2

| | % w/w |
|---|---|
| β-carotene (30% dispersion) | 15.5 |
| emulsifier (polysorbate 80) | 82 |
| emulsifier (Span 80) | 2.5 |

The β-carotene is dispersed in the two emulsifiers and the mixture is heated to 140° C. with stirring. At this point the mixture should remain transparent. Finally, the mixture is cooled rapidly to room temperature.

The resultant mixture is encapsulated in gelatin capsules so as to contain approx. 15 mg β-carotene per capsule.

### EXAMPLE 3

| | % w/w |
|---|---|
| tocopheryl acetate | 11 |
| emulsifier (polysorbate 40) | 44.5 |
| polyethylene glycol | 44.5 |

The tocopheryl acetate is dispersed in the emulsifier and polyethylene glycol with stirring at room temperature. The mixture should remain transparent.

The resultant mixture is encapsulated in gelatin capsules so as to contain approx. 15 mg tocopheryl acetate per capsule.

### EXAMPLE 4

| | % w/w |
|---|---|
| β-carotene (crystalline) | 12.8 |
| emulsifier (polysorbate 60) | 77.0 |
| emulsifier (sugar ester S-1170) | 3.2 |
| emulsifier (Triodan) | 3.2 |
| α-tocopherol (antioxidant) | 3.8 |

α-Tocopherol is dispersed in a mixture of the three emulsifiers, followed by the β-carotene and the mixture is heated to 140° C. with stirring. At this point the mixture should remain transparent. Finally, the mixture is cooled rapidly to room temperature.

The resultant mixture is encapsulated in gelatin capsules so as to contain approx. 15 mg β-carotene per capsule.

Bioavailability Study Protocol

Capsule formulations of this invention can be tested to assess improved bioavailability as follows:

A comparative study to demonstrate the appearance of beta-carotene in blood serum following the ingestion of 15 mg doses either in the form described by the invention or in a commercially available form (Roche Redoxon). The study is designed to give information on the initial rate of uptake and amplitude of response for each dosage form.

Subjects:

20 subjects, male and female. Age range 15-50 years. Volunteers with any history or biochemical evidence of liver, kidney, or pancreatic disease, anaemia, hyperlopidaemia or malabsorption syndromes are excluded. Subjects taking vitamin, beta-carotene or other supplements and medications suspected of interfering with the absorption of fat-soluble actives are excluded. Also excluded are individuals on a vegetarian diet, those not following normal dietary practices, subjects with weights greater or less than 20% of the ideal for their height, age and sex, smokers, pregnant females, females taking oral contraceptives and those whose diet contains greater than an estimated daily intake of 3.5 mg beta-carotene per day.

Diet:

All subjects will be required to complete dietary questionnaires prior to the study to identify dietary fat and beta-carotene intakes. They are allowed a free choice of diet during the study except for the breakfast prior to dosing. The prescribed breakfast is a choice of non-maize based cereal with skimmed or semi-skimmed milk, yoghurt either plain or red fruit varieties, coffee or tea with skimmed or semi-skimmed milk, non-carotenoid containing fruit juice or conserves. Fat intake must be avoided. At other times, a normal diet is to be followed but with the exclusion of incidences of high fat intake e.g cream, and foods containing high concentration s of beta-carotene.

Test materials:

Capsules containing 15 mg beta-carotene, either test material according to the invention or Roche Products Redoxon capsules. Placebo capsule containing corn oil. Capsules to be swallowed with 250 ml water.

Experimental Design:

| Time (hours) | |
|---|---|
| −48 | Blood sample |
| −1 | Breakfast |
| 0 | Blood sample followed by dose 1 |
| +2 | Blood sample |
| +4 | Blood sample |
| +8 | Blood sample |
| +23 | Breakfast |
| +24 | Blood sample followed by dose 2 |
| +47 | Breakfast |
| +48 | Blood sample followed by dose 3 |
| +71 | Breakfast |
| +72 | Blood sample followed by dose 4 |
| +95 | Breakfast |
| +96 | Blood sample followed by dose 5 |
| +119 | Breakfast |
| +120 | Blood sample followed by dose 6 |
| +143 | Breakfast |
| +144 | Blood sample followed by dose 7 |
| +168 | Blood sample |

4 weeks after the completion of this leg, the design is repeated for each subject with the second test material. The order in which the regimens are undertaken are varied for each subject.

Sampling and analysis:

10 ml samples of blood are collected by means of an indwelling catheter inserted in forearm vein on day 1 of the regime; thereafter samples are withdrawn by means of a syringe. Following separation of the serum, the serum samples are stored at -24° C. for later analysis by high-pressure liquid chromatography.

All publications, including but not limited to patents and patent applications, cited in this specification are herein incorporated by reference as if each individual publication were specifically and individually indicated to be incorporated by reference herein as though fully set forth.

The above description fully discloses the invention including preferred embodiments thereof. Modifications and improvements of the embodiments specifically disclosed herein are within the scope of the following claims. Without further elaboration, it is believed that one skilled in the are can, using the preceding description, utilize the present invention to its fullest extent. Therefore, the Examples herein are to be construed as merely illustrative and not a limitation of the scope of the present invention in any way. The embodiments of the invention in which an exclusive property or privilege is claimed are defined as follows.

What is claimed is:

1. An essentially non-aqueous composition for oral administration comprising a biologically active oil, or a biologically active oil or an oil-soluble ingredient as a dispersion in a suitable carrier oil, dispersed in a physiologically acceptable emulsifier having an HLB value in the range 10 to 18 and encapsulated in a capsule material that is soluble in gastric juice, wherein the emulsifier is an emulsifier mixture comprising a combination of a primary surfactant and a cosurfactant wherein the fatty acid profile of the emulsifier mixture matches the fatty acid profile of the oil component of the composition, the HLB of the primary surfactant is greater than that of the cosurfactant and the ratio of primary surfactant to cosurfactant is in the range 10:1 to 200:1.

2. A composition as claimed in claim 1 in which the weight ratio of total oil to emulsifier is in the range 1:1 to 1:7.

3. A composition as claimed in claim 1 in which the emulsifier is a non-ionic emulsifier having an HLB in the range 12 to 16.

4. A composition as claimed in claim 1 comprising 0.15-50% w/w of a biologically active oil, or a biologically active oil or an oil-soluble ingredient as a dispersion in a suitable carrier oil, and 50-99.85% w/w of emulsifier.

5. A composition as claimed in claim 1 comprising an antioxidant.

6. A composition as claimed in claim 1 comprising a biologically active oil of natural origin.

7. A composition as claimed in claim 1 comprising an oil-soluble ingredient which is a carotenoid or oil-soluble vitamin.

8. A composition as claimed in claim 1 in which the capsule material is gelatin.

9. A process for preparing a composition as claimed in claim 1 comprising mixing the biologically active oil, or dispersion of the biologically active oil or oil-soluble ingredient in a suitable carrier oil, with the emulsifier mixture so as to yield a transparent mixture, at elevated temperature if required, and encapsulating the resultant transparent mixture in a capsule material.

10. A method of improving the bioavailability of a biologically active oil or oil-soluble ingredient in a human or animal in need thereof which method comprises orally administering to said human or animal a composition as defined in claim 1.
